(19) United States
(12) Patent Application Publication
(10) Pub. No.: US 2025/0031986 A1
(43) Pub. Date: Jan. 30, 2025

(54) SYSTEM AND METHOD FOR ADJUSTING HEART RATE MEASUREMENT ACCURACY OF A WEARABLE DEVICE

(71) Applicant: SAMSUNG ELECTRONICS CO., LTD., Suwon-si (KR)

(72) Inventors: Rohan CHOUDHARY, Noida (IN); Deepak SINGH, Noida (IN)

(73) Assignee: SAMSUNG ELECTRONICS CO., LTD., Suwon-si (KR)

(21) Appl. No.: 18/784,591

(22) Filed: Jul. 25, 2024

Related U.S. Application Data
(63) Continuation of application No. PCT/KR2024/006130, filed on May 8, 2024.

(30) Foreign Application Priority Data
Jul. 28, 2023 (IN) 202311051098

(51) Int. Cl. A61B 5/024; A61B 5/00; A61B 5/11; G16H 10/60; G16H 40/67

(52) U.S. Cl. CPC A61B 5/02433; A61B 5/1118; A61B 5/4266; A61B 5/443; A61B 5/681; A61B 5/7225; G16H 10/60; G16H 40/67; A61B 2560/0252; A61B 2562/029

(57) ABSTRACT

A method for adjusting heart rate measurement accuracy of a wearable device includes: obtaining, by a data module, a plurality of pieces of data including user activity data, user information, and environment conditions related to a user from a plurality of data sources; determining a skin attribute of the user based on the obtained plurality of pieces of data; emitting, by a transmitter module, infrared (IR) radiation and visible light at an intensity level for measuring a heart rate of the user; receiving, by a receiver module, scattered IR radiation resulting from interaction of the transmitted IR radiation and the transmitted visible light based on a skin condition of the user and the environment conditions; determining, by a correlation module, a scattered radiation vector based on the scattered IR radiation; determining, by a detection module, a wavelength of the visible light to be adjusted based on the scattered radiation vector; and adjusting, by the transmitter module, the wavelength of the visible light.

### CROSS-REFERENCE TO RELATED APPLICATION(S)

This application is a continuation application, claiming priority under § 365(c), of an International application No. PCT/KR2024/006130, filed on May 8, 2024, which claims priority under 35 U. S. C. § 119 to Indian Patent Application No. 202311051098, filed on Jul. 28, 2023, the disclosures of which are incorporated herein by reference in their entireties.

### BACKGROUND

### 1. Field

The disclosure relates to heart rate measurement, and more particularly, to a system and method for improving heart rate measurement accuracy of a wearable device.

### 2. Description of Related Art

Heart rate provides insight into the overall health of the heart. Over time, an increased heart rate may indicate various conditions, such as coronary artery disease and heart valve problems. Detection of these conditions at an early stage may be beneficial for intervening and offering treatment to protect the heart from damage. Further, heart rate is an important vital signs for monitoring health, tracking exercise, and guiding recovery procedures for users. Therefore, a routine heart rate measurement may be useful for monitoring cardiovascular disease treatment progress or an overall health of a user.

Various options are available for measuring the heart rate, for example, a photoplethysmography (PPG) sensor in a wearable device such as a smart watch. Although the smart watch provides an easy and convenient way to monitor the heart rate, the readings are often inaccurate due to an inability of the device to consider different environmental conditions and factors such as skin attributes, watch fit, physical activity, gender, and age. These inaccuracies may pose a significant challenge to healthcare professionals and results in misleading diagnoses of conditions such as hypertension, arrhythmias, or heart disease. Further, inaccurate readings may also impose negative effects on an individual's mental health, leading to worry and anxiety.

Therefore, there is a need for a system or method that can adequately consider these environmental conditions and factors during heart rate measurement, and improve the heart rate measurement accuracy of the wearable device.

Some approaches for heart rate measurement relate to an optical device for determining pulse rate, which may be for example included in a wearable monitoring device which includes a motion sensor and a PPG sensor. For example, the PPG sensor may include a periodic light source, a photo detector, and circuitry for determining a heart rate of a user based on an output of the photo detector. However, these approaches may not consider skin attributes of the user corresponding to an activity or an environment of the user. Further, other approaches may relate to a continuous heart rate monitoring and interpretation, for example by selecting from among two or more different modes for heart rate detection and may provide automated recommendations concerning changes to sleep, recovery time, exercise routines and the like. However, these approaches may not consider skin attributes of the user corresponding to an activity or an environment of the user.

Therefore, there is a need to overcome the aforementioned drawbacks associated with these approaches for improving heart rate measurement accuracy of a wearable device.

### SUMMARY

According to an aspect of the disclosure, a method for adjusting heart rate measurement accuracy of a wearable device, includes: obtaining, by a data module, a plurality of pieces of data including user activity data, user information, and environment conditions related to a user from a plurality of data sources; determining a skin attribute of the user based on the obtained plurality of pieces of data; emitting, by a transmitter module, infrared (IR) radiation and visible light at an intensity level for measuring a heart rate of the user; receiving, by a receiver module, scattered IR radiation resulting from interaction of the transmitted IR radiation and the transmitted visible light based on a skin condition of the user and the environment conditions; determining, by a correlation module, a scattered radiation vector based on the scattered IR radiation; determining, by a detection module, a wavelength of the visible light to be adjusted based on the scattered radiation vector; and adjusting, by the transmitter module, the wavelength of the visible light.

The user activity data may include physical activity data of the user, the user information may include at least one of electronic health record data, disease information, age, gender, and location of the user, and the environment conditions may include a temperature and a humidity of an environment of the user.

The plurality of data sources may include at least one of an electronic health record data of the user, a photoplethysmography sensor, a temperature sensor, an accelerometer, and a gyroscope.

The method may further include: determining, using an input module included in the data module, user demographics based on the user information obtained from the plurality of data sources; and determining, using a skin attribute extraction module included in the data module, the skin attribute based on at least one of the user demographics obtained from the input module, and electronic health record data, physical activity data, temperature data, and humidity data obtained from the plurality of data sources.

The determining the skin attribute of the user may include: determining, using a skin type vector determining sub-module, a skin type vector of the user based on at least one of the electronic health record data and historical data provided by the user, wherein the skin type vector indicates at least one of a dry skin magnitude, an oily skin magnitude, and a normal skin magnitude based on the skin condition of the user; determining, by a sweat amount determining sub-module, a sweat amount based on the user demographics, the physical activity data, the temperature data, and the humidity data using a machine learning model; and determining, by a vector merger, the skin attribute by merging the skin type vector determined by the skin type vector determining sub-module and the sweat amount determined by the sweat amount determining sub-module.

The user demographics, the plurality of pieces of data, and the skin attribute of the user may be stored in a database.

The method may further include: receiving, using a controller included in the transmitter module, input from the data module and the detection module and determining the intensity level of the visible light based on a red light coefficient and a green light coefficient, wherein the visible light includes red light and green light; and emitting, using a modulator included in the transmitter module, IR radiation along with the green light and the red light based on the determined intensity level.

The method may further include at least one of: separating different wavelengths of light using a wavelength division multiplexing (WDM) splitter; and combining the different wavelengths of light into one output channel using a WDM combiner.

The method may further include: detecting the scattered IR radiation and converting the scattered IR radiation into an electrical signal using a photo detector; amplifying the electrical signal using a preamplifier; filtering the electrical signal based on filter coefficients using a filter, wherein the filter coefficients depend on the wavelength of the visible light; improving a quality of the electrical signal using at least one of an amplifier and a limiter; improving a signal-to-noise ratio, reducing a distortion, and minimizing an interference of the electrical signal using a gain controlling sub-module; processing the electrical signal using a decision circuit may include at least one of a low-pass filter and a comparator; and providing the electrical signal to the correlation module.

The determining the scattered radiation vector may include: receiving, by a photoplethysmography sensor, the scattered IR radiation from the receiver module and determining amount of absorbed IR radiation and an amount of absorbed visible light, wherein the absorbed visible light may include at least one of green light and red light; and determining, by a radiation vector creation sub-module, the scattered radiation vector and identifying a deviation by correlating an amount of IR radiation absorption and visible light absorption with similar wavelength absorption corresponding to a plurality of skin attributes stored in a database using a machine learning model.

The method may further include determining, by the detection module, a red light coefficient and a green light coefficient based on the scattered radiation vector; and determining the intensity level of the visible light based on the determined red light coefficient and the determined green light coefficient using a coefficient generation neural network.

According to an aspect of the disclosure, a system for adjusting heart rate measurement accuracy of a wearable device, includes: a data module configured to obtain a plurality of pieces of data may include user activity data, user information, and environment conditions from a plurality of data sources and determine a skin attribute of a user based on the obtained plurality of pieces of data; a transmitter module configured to emit infrared (IR) radiation along with visible light at an intensity level for measuring a heart rate of the user; a receiver module configured to receive scattered IR radiation resulting from interaction of the transmitted IR radiation and the transmitted visible light with based on a skin condition of the user and the environment conditions; a correlation module configured to determine a scattered radiation vector based on the scattered IR radiation; and a detection module configured to determine wavelength of the visible light to be adjusted based on the scattered radiation vector,
wherein the transmitter module is further configured to adjust the wavelength of the visible light.

The user activity data may include user physical activity data, the user information may include at least one of electronic health record data, disease information, age, gender, and location of the user, and the environment conditions may include at least one of a temperature and a humidity of an environment of the user.

The plurality of data sources may include at least one of an electronic health record data of the user, a photoplethysmography sensor, a temperature sensor, an accelerometer, and a gyroscope.

According to an aspect of the disclosure, a non-transitory computer readable recording medium stores instructions which, when executed by at least one processor of a device for improving heart rate measurement accuracy of a wearable device, cause the device to: obtain, by a data module, a plurality of pieces of data may include user activity data, user information, and environment conditions related to a user from a plurality of data sources; determine a skin attribute of the user based on the obtained plurality of pieces of data; emit, by a transmitter module, infrared (IR) radiation along with visible light at an intensity level for measuring heart rate of the user; receive, by a receiver module, scattered IR radiation resulting from interaction of the emitted IR radiation and the emitted visible light based on a skin condition of the user and the environment condition; determine, by a correlation module, a scattered radiation vector based on the scattered IR; determine, by a detection module, a wavelength of the visible light to be adjusted based on the scattered radiation vector; and adjust, by the transmitter module, the wavelength of the visible light.

The foregoing summary is illustrative only and is not intended to be in any way limiting. In addition to the illustrative aspects, embodiments, and features described earlier, further aspects, embodiments, and features will become apparent by reference to the drawings and the following detailed description.

### BRIEF DESCRIPTION OF THE DRAWINGS

The above and other aspects, features, and advantages of certain embodiments of the present disclosure will be more apparent from the following description taken in conjunction with the accompanying drawings, in which:

FIG. 1 depicts a flow diagram showing a method for improving heart rate measurement accuracy of a wearable device, in accordance with one or more embodiments;

FIG. 2 depicts a block diagram of the system performing method for improving heart rate measurement accuracy of the wearable device, in accordance with one or more embodiments;

FIG. 3 depicts a block diagram of a data module, in accordance with one or more embodiments;

FIG. 4 depicts a block diagram of a skin attribute extraction module, in accordance with one or more embodiments;

FIG. 5 depicts an exemplary embodiment of determining sweat amount by a sweat amount determining sub-module, in accordance with one or more embodiments;

FIG. 6 depicts a flow diagram showing a method for determining skin attributes, in accordance with one or more embodiments

FIG. 7A depicts a block diagram of a transmitter module, in accordance with one or more embodiments;

FIG. 7B depicts a pictorial representation of wavelength division multiplexing utilized by the transmitter module, in accordance with one or more embodiments;

FIG. 8 depicts a block diagram of a receiver module, in accordance with one or more embodiments;

FIG. 9 depicts a block diagram of a correlation module, in accordance with one or more embodiments;

FIG. 10 depicts a flow diagram showing a method of determining scattered radiation vector, in accordance with one or more embodiments;

FIG. 11A depicts a first use case of improving heart rate measurement accuracy of the wearable device, in accordance with one or more embodiments; and

FIG. 11B depicts a second use case of improving heart rate measurement accuracy of the wearable device, in accordance with one or more embodiments.

### DETAILED DESCRIPTION

In the following description, numerous specific details are set forth in order to provide a thorough understanding of the present disclosure. It will be apparent, however, to one skilled in the art that these specific details are only examples and not intended to be limiting. Additionally, it may be noted that the systems and/or methods are shown in block diagram form only in order to avoid obscuring the present disclosure. It is to be understood that various omissions and substitutions of equivalents may be made as circumstances may suggest or render expedient to cover various applications or implementations without departing from the spirit or the scope of the present disclosure. Further, it is to be understood that the phraseology and terminology employed herein are for the purpose of clarity of the description and should not be regarded as limiting.

Furthermore, in the present description, references to “one embodiment” or “an embodiment” means that a particular feature, structure, or characteristic described in connection with the embodiment is included in at least one embodiment of the present disclosure. The appearance of the phrase “in one embodiment” in various places in the specification is not necessarily referring to the same embodiment, nor are separate or alternative embodiments mutually exclusive of other embodiments. Further, the terms “a” and “an” used herein do not denote a limitation of quantity, but rather denote the presence of at least one of the referenced items. Moreover, various features are described which may be exhibited by some embodiments and not by others. Similarly, various requirements are described, which may be requirements for some embodiments but not for other embodiments.

Wearable devices such as smartwatches offer a convenient and accessible means of monitoring an individual's heart rate. However, these devices don't provide accurate heart rate measurements, particularly under varying environmental conditions and factors. Inaccuracies in these measurements generally occur due to environment condition and factors such as skin attributes, watch fit, physical activity, gender, and age.

Improving heart rate measurement accuracy of these wearable devices may be beneficial for achieving reliable and valid readings that can be used for various clinical, research, and personal purposes.

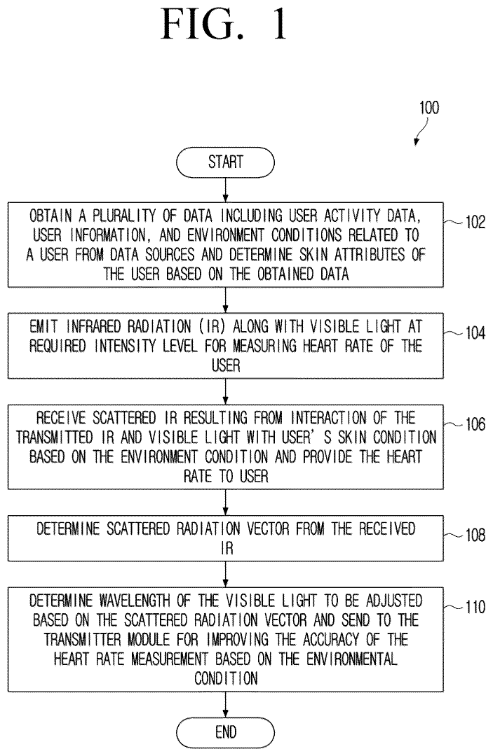

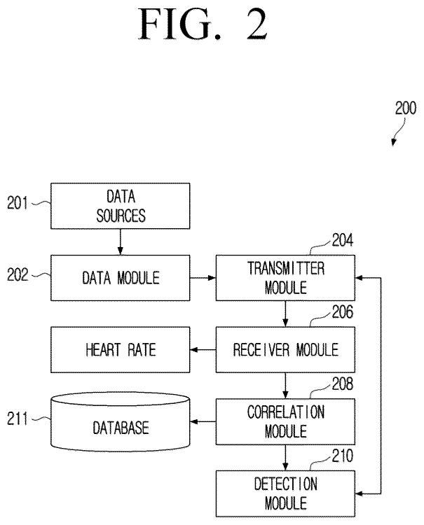

FIG. 1 shows a flow diagram showing a process 100 for improving heart rate measurement accuracy of a wearable device, according to one or more embodiments. The method may be explained in conjunction with the system illustrated in FIG. 2. In the flow diagram, each block may represent a module, segment, or portion of code, which may include one or more executable instructions for implementing the specified logical function or functions. It should also be noted that in some implementations, the function or functions illustrated as being included in the blocks may occur out of the order noted in the drawings. For example, two blocks shown in succession in FIG. 1 may be executed substantially concurrently, or the blocks may sometimes be executed in the reverse order, depending upon the functionality involved. Any process descriptions or blocks in flowcharts should be understood as representing modules, segments, or portions of code that include one or more executable instructions for implementing specific logical functions or steps in the process, and alternate implementations are included within the scope of the example embodiments in which functions may be executed out of order from that shown or discussed, including substantially concurrently or in reverse order, depending on the functionality involved. In addition, the process descriptions or blocks in flow charts should be understood as representing decisions made by a hardware structure such as a state machine.

As shown in FIG. 1, the process 100 may begin at operation 102, and proceed through operation 110.

At operation 102, a plurality of pieces of data including user activity data, user information, and environment conditions related to a user may be obtained from data sources, for example data sources 201, and skin attributes of the user may be determined based on the obtained data. In one embodiment, the user activity data may include data about a physical activity data of the user, such as the number of steps taken, duration of physical activity, and intensity of the activity. The user information may include, but is not limited to, information from at least one electronic health record, disease information, age, gender, and location of the user, and the environment conditions may include, but is not limited to, temperature and humidity of the environment in which the user is present. The plurality of pieces of data may be obtained from the data sources 201 may include electronic health record data 306 of the user, and data from a photoplethysmography (PPG) sensor 308, a temperature sensor 310, an accelerometer 312, and a gyroscope 314.

Next, infrared (IR) radiation along with visible light may be emitted at operation 104. In one embodiment, the IR radiation along with the visible light, which may include red light and green light at a suitable intensity level, may be emitted for measuring heart rate of the user. The transmitter module 204 may use wavelength division multiplexing (WDM) splitters or combiners to separate different wavelengths of light and combine different wavelengths of light into one output channel, respectively.

Next, scattered IR radiation resulting from interaction of the transmitted IR radiation and visible light with based on a skin condition of the user and the environment condition may be received at operation 106. It should be noted that the scattering of IR radiation may occur because the skin absorbs and reflects different wavelengths of light to varying degrees, depending on factors like skin pigmentation, blood flow, and environment conditions such as temperature and humidity. By analyzing the scattered IR radiation, the wearable device may provide an accurate heart rate measurement.

Next, a scattered radiation vector corresponding to the received IR radiation may be determined at operation 108. In one embodiment, the scattered radiation vector may be determined by a machine learning model which compares amount of IR radiation absorption and visible light absorption determined from the scattered IR radiation with similar wavelength absorption from a database 211 to identify the deviation. The scattered radiation vector may represent user attributes such as attributes of skin of a user during physical activity. In an embodiment, the scattered radiation vector may provide information such as the presence of sweat due to light walking or sweat due to jogging performed by the user of dry skin.

Thereafter, wavelength of the visible light to be adjusted may be determined and sent to the transmitter module 204, at operation 110. In one embodiment, wavelength of the visible light to be adjusted may be determined based on the scattered radiation vector and sent to the transmitter module 204 to improve the accuracy of the heart rate measurement based on the environmental condition.

FIG. 2, is a block diagram of a system 200 for improving heart rate measurement accuracy of the wearable device, in accordance with one or more embodiments. In an embodiment, the wearable device may include, but is not limited to, fitness trackers, wristbands, wristwatches, augmented reality glasses, smart glasses, or any other electronic device that is worn, implanted, or embedded on the body. In embodiments, the system 200 may be, may include, or may be included in the wearable device, but embodiments are not limited thereto.

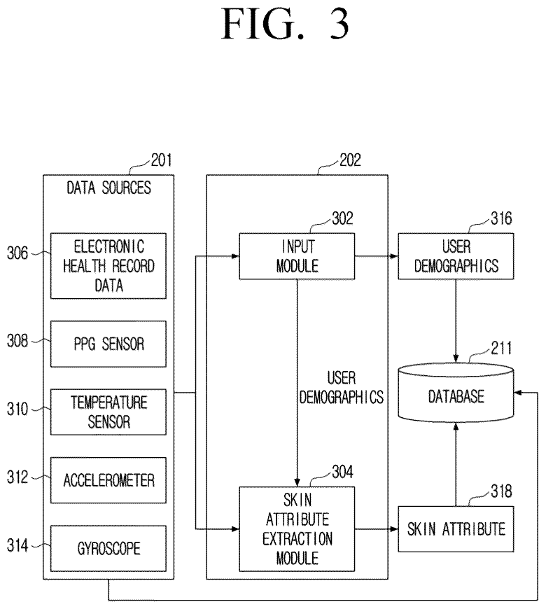

The system 200 may include a data module 202 for obtaining a plurality of pieces of data including user activity data, user information, and environment conditions from data sources, for example data sources 201. The user activity data may include data about a physical activity of the user. The user information may include, but is not limited to, information from at least one electronic health record, disease information, age, gender, and location of the user. The environment conditions may include, but are not limited to, temperature and humidity of the environment in which the user is present. It should be noted that the data module 202 may be configured to obtain data used for measuring the accurate heart rate. The data module 202 may be further configured to determine skin attributes of the user based on the obtained data, an example of which is described below with respect to FIG. 3.

FIG. 3, is a block diagram of illustrating an example of the data module 202, in accordance with one or more embodiments. As depicted, the data module 202 may include an input module 302 and a skin attribute extraction module 304 and may be configured to obtain the plurality of pieces of data from the data sources 201 including electronic health record data 306 of the user, and data from a PPG sensor 308, a temperature sensor 310, an accelerometer 312, and a gyroscope 314. It should be noted that the electronic health record data 306 may be obtained from various sources, including healthcare facilities such as hospitals, primary care facilities, and rehabilitation centers and are integrated into an electronic health record system for providing a holistic view of a medical history of the user. The PPG sensor 308 may be configured to detect changes in blood flow and sweat, the temperature sensor 310 may be configured to measure changes in skin temperature and sweat, the accelerometer 312 may be configured to detect user activity and monitoring fit of the watch, and the gyroscope 314 may be is configured to determine an orientation of the wearable device.

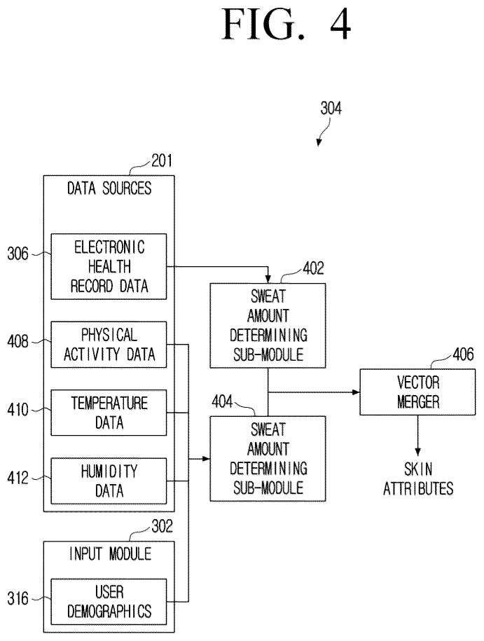

In one embodiment, the input module 302 may be configured to determine user demographics 316 from the user information obtained from the data sources and the skin attribute extraction module 304 may be configured to determine skin attributes 318 using the user demographics from the input module 302 and/or physical activity data 408, the temperature data 410 and humidity data 412 from the data sources 201, examples of which are explained in detail below with reference to FIG. 4. The user demographics 316 from the input module 302, the skin attributes 318 of the user from the skin attribute extraction module 304 and a plurality of pieces of data from the data sources 201 may be stored in a database 211. In an embodiment, the database 211 may store information such as skin temperature, time of current physical activity, calories burned in the current physical activity, humidity, age, gender, weight, height, sweat amount, and other relevant metrics.

FIG. 4 is a block diagram of the skin attribute extraction module 304, in accordance with one or more embodiments. As shown in FIG. 4, the skin attribute extraction module 304 may include a skin type vector determining sub-module 402 for determining a skin type vector of the user using electronic health record data 306 from the data sources 201 and/or historical data provided by the user. In an embodiment, the electronic health record data may incorporate a comprehensive medical history of the user, including data related to allergies, clinical vital signs, overall health condition, diagnoses and its associated medication, laboratory results, medical procedures undergone, and other relevant medical data. The skin type vector may include or indicate dry skin magnitude, oily skin magnitude, and normal skin magnitude based on dry skin and oily skin conditions. The dry skin conditions may include eczema, psoriasis, xerosis, ichthyosis, and other similar conditions that may cause the skin to become itchy, flaky, or cracked, while the oily skin conditions may include acne, seborrheic dermatitis, contact dermatitis, and other related conditions that may cause the skin to appear greasy or oily. In an embodiment, the skin type vector may include three dimensions such as dry skin magnitude, oily skin magnitude, and normal skin magnitude, as shown in Table 1 below:

TABLE 1
Skin type vector based on different skin conditions

| User | Dry Skin | Dry Skin | Oily Skin | Oily Skin | Normal | Skin Type |
|---|---|---|---|---|---|---|
| No. | Conditions | Magnitude | Conditions | Magnitude | Skin | Vector |
| 1 | Ichthyosis, | 0.9 + | Contact | 0.3 | 1 − 1.5 − | [1.5, 0.3, 0] |
| | Xerosis | 0.6 = 1.5 | Dermatitis | | 0.3 = −0.8 => 0 | |
| 2 | Psoriasis, | 0.8 + | None | 0 | 1 − 1.5 − | [1.5, 0, 0] |
| | Eczema | 0.7 = 1.5 | | | 0 = −0.6 => 0 | |
| 3 | None | 0 | Seborrheic | 0.9 | 1 − 0 − | [0, 0.9, 1] |
| | | | Dermatitis | | 0.9 = 0.1 => 0.1 | |
| 4 | None | 0 | None | 0 | 1 − 0 − | [0, 0, 1] |
| | | | | | 0 = 1 => 1 | |
| 5 | Xerosis, | 0.6 + 0.7 + | None | 0 | 1 − 2.1 − | [2.1, 0, 0] |
| | Eczema, | 0.8 = 2.1 | | | 0 = −1.1 => 0 | |
| | Psoriasis | | | | | |
| 6 | Xerosis | 0.6 | Acne | 0.7 | 1 − 0.6 − | [0.6, 0.7, 0] |
| | | | | | 0.7 = −0.3 => 0 | |

As shown in table 1, the skin type vector may be dependent on the magnitudes of dry, oily, and normal skin conditions. The dry skin magnitude may be the sum of coefficients of all dry skin conditions that the user is experiencing, while the oily skin magnitude may be the sum of coefficients of all the oily skin conditions the user is experiencing. The normal skin magnitude depends on the dry and oily skin magnitudes, and is computed using the Equation 1 below:

$$Equation 1$$

$$Normal skin magnitude = [ 1 - dry skin magnitude - oily skin magnitude ]$$

If the normal skin magnitude computed using this formula results in a negative value, then the normal skin magnitude may be set to “0”.

It should be noted that each skin magnitude may be computed from coefficients of respected skin conditions. In an embodiment, a coefficient for the dry skin conditions, such as ichthyosis, may be 0.9, since the skin condition ichthyosis may cause severe dryness, roughness, scaling, and thickening of the skin. The coefficient for psoriasis may be 0.8, due to significant dryness caused by this type of skin condition, while the coefficient for the eczema may be 0.7, considering moderate dryness caused by this condition. The coefficient for the xerosis may be 0.6, considering the condition causes noticeable but not severe dryness and flakiness.

Further, coefficient for oily skin conditions, such as seborrheic dermatitis, may be 0.9, because this skin condition causes excessive oil production. The coefficient for acne may be 0.7, due to moderate amount of oil production, while the coefficient for contact dermatitis may be 0.3, considering the condition causes comparatively less oil production.

The skin attribute extraction module 304 may further include a sweat amount determining sub-module 404 for determining a sweat amount based on user demographics 316 from the input module 302 and the physical activity data 408, the temperature data 410 and the humidity data 412 from the data sources 201 by utilizing a machine learning model. In an embodiment, the heart rate measurement obtained using the PPG sensor 308 may be directly dependent on humidity, physical activity, and temperature, because these factors may affect the skin and further lead to distortions in PPG reading.

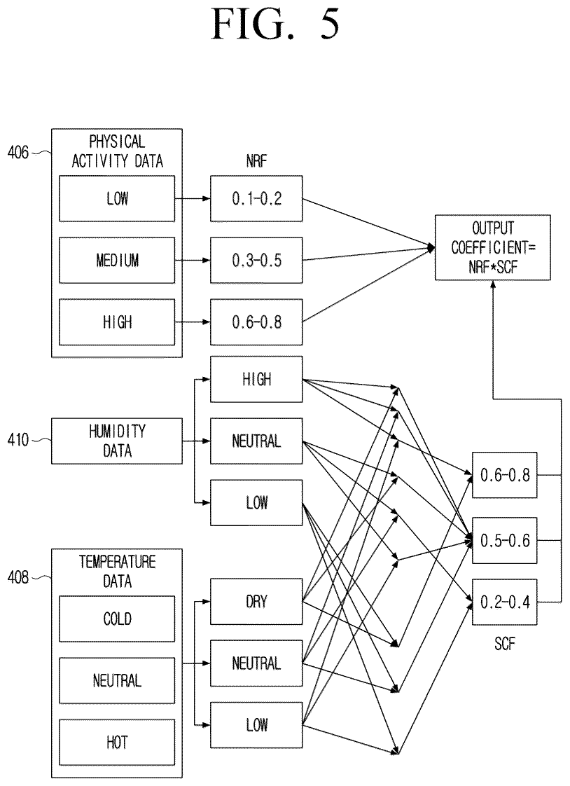

In embodiments, low humidity and low temperatures may lead to a decrease in skin barrier function, making the skin more susceptible to mechanical stress and increasing the release of pro-inflammatory cytokines and cortisol from keratinocytes. These conditions may lead to an increase in the number of dermal mast cells, consequently causing the skin to become more reactive to skin irritants and allergens. Therefore, the sweat amount determining sub-module 404 may use a machine learning model for determining the sweat amount, as depicted in FIG. 5.

In an embodiment, the skin may turn red due to sweat under certain humidity and temperature conditions, this color change can affect the PPG readings. The machine learning model may be used to detect such conditions and adjust the readings such that the color change does not affect the actual heart rate measurement.

FIG. 5, illustrates an embodiment of determining the sweat amount by the sweat amount determining sub-module. As shown in FIG. 5, a noise reduction factor (illustrated as “NRF”) may be determined for the physical activity, which may be for example one of low, medium, and high. In one embodiment, the sweat amount determining sub-module 404 may use a machine learning model to determine the noise reduction factor. In another embodiment, the noise reduction factor may be user-defined. Furthermore, the skin condition factor (illustrated as “SCF”) may be determined by the sweat amount determining sub-module 404 using the machine learning model for received temperature data 410 and humidity data 412. In an embodiment, the skin condition factor may be determined based on the type of humidity, such as high, neutral, and low, and type of temperature, such as cold, neutral, and hot, which may correspond respectively to dry, neutral, and sweat.

After determining both the noise reduction factor and skin condition factor, the sweat amount determining sub-module 404 may use both factors to accurately calculate the amount of sweat present on the skin.

The skin attribute extraction module 304 may further include a vector merger 406 for determining the skin attributes 318 by merging the skin type vector received from the skin type vector determining sub-module 402 and the sweat amount received from the sweat amount determining sub-module 404. In an embodiment, the vector merger 406 may use a machine learning model to determine the skin attributes 318 containing information about sweat and skin. It should be noted that value of each dimension of the skin type vector and sweat amount may be in a range between 0 to 1, as shown in Table 2 below:

TABLE 2
Different skin attributes for different
skin type vector and sweat amount.

| Skin Type | Sweat | | |
|---|---|---|---|
| Vector | Amount | Skin Attributes | Remarks |
| 0.8 | [0.3] | [0.8, 0.3] | Sweat factor overruled the |
| | | | dry factor as the skin is oily |
| 0.6 | [0.6] | [0.6, 0.6] | Sweat and dry factor both |
| | | | needs to be considered |
| 0.4 | [0.2] | [0.4, 0.2] | Dry factor overruled sweat |
| | | | factor as skin is dry and |
| | | | sweat amount is less |
| 0.7 | [0.8] | [0.7, 0.9] | Sweat factor overruled the |
| | | | dry factor as skin is oily |

As shown in Table 2, the skin attributes 318 may include both skin type vector and sweat amount. For example, when the skin type vector is 0.8, indicating highly oily skin, and the sweat amount is 0.3, the skin attribute 318 may indicate that the sweat factor overrules or invalidates the dry factor due to the presence of oil on the skin. As another example, when the skin type vector is 0.6, indicating oily skin, and the sweat amount is 0.6, the skin attribute may indicate that both the sweat and dry factor should be considered. As yet another example, when the skin type vector is 0.4, indicating dry skin, and the sweat amount is 0.2, the skin attribute may indicate that the dry factor overrules or invalidates the sweat factor due to dryness of the skin and the low sweat amount. As yet another example, when the skin type vector is 0.7, indicating highly oily skin, and the sweat amount is 0.8, the skin attribute may indicate that the sweat factor overrules or invalidates the dry factor due to the presence of oil on the skin.

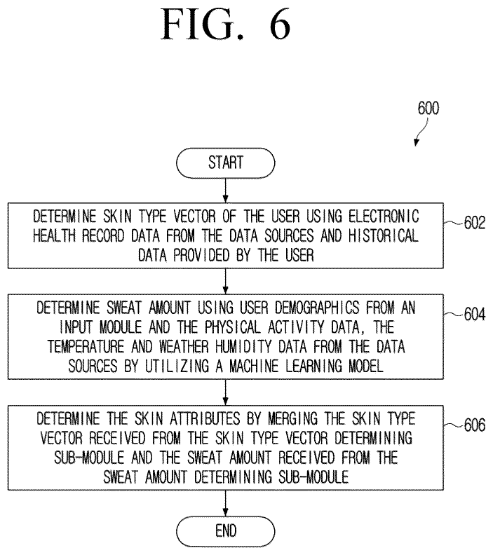

Referring to FIG. 6, a method for determining the skin attributes is depicted. As shown in FIG. 6, the skin type vector of the user may be determined at operation 602 using at least one of electronic health record data 306 from the data sources 201 and historical data provided by the user. In one embodiment, the skin type vector may include at least one of dry skin magnitude, oily skin magnitude, and normal skin magnitude based on skin conditions, for example dry skin conditions and oily skin conditions. Next, sweat amount may be determined at operation 604 using user demographics 316 from the input module 302 and the physical activity data 408, the temperature data 410 and the humidity data 412 from the data sources 210 by using a machine learning model. Thereafter, the skin attributes 318 may be determined at operation 606, by merging the skin type vector received from the skin type vector determining sub-module 402 and the sweat amount received from the sweat amount determining sub-module 404.

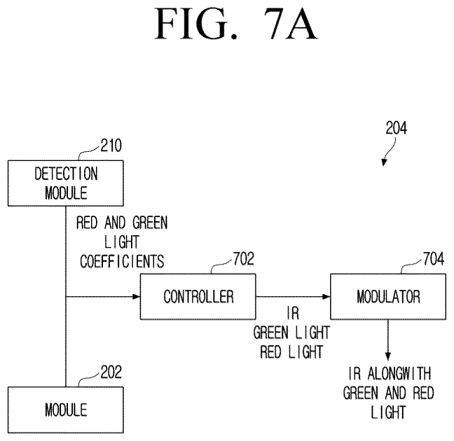

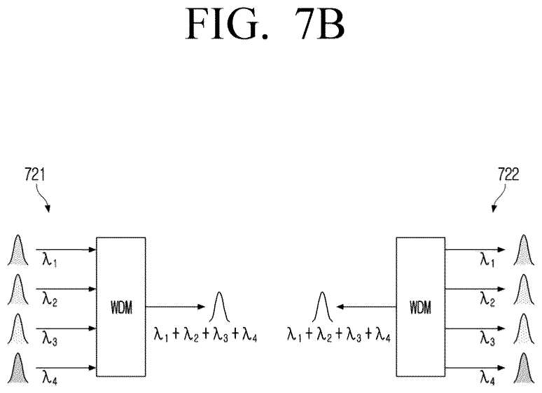

Referring again to FIG. 2, the system 200 may further include a transmitter module 204 configured to emit IR radiation along with visible light at a suitable intensity level for measuring a heart rate of the user. In one embodiment, the transmitter module 204 may include a plurality of sub-modules as depicted in FIG. 7A.

FIG. 7A, is a block diagram of the transmitter module 204, in accordance with one or more embodiments. The transmitter module 204 may include a controller 702 for receiving input from the data module 202 and an detection module 210 and determining a suitable intensity level of the visible light based on red light coefficient and green light coefficient. In one embodiment, the visible light includes red light and green light. It should be noted that the green light has a shorter wavelength, higher frequency, and greater energy in comparison to the red light, which may allow it to penetrate more deeply into the skin's pores than red light. Therefore, green light may be used for operations corresponding to deeper layers of the skin, whereas red light may be employed for operation affecting the surface level of the skin. The controller may be configured to operate based on the skin conditions. The transmitter module 204 may further include a modulator 704 for performing adjustment of amplitudes, frequencies, or phases of infrared radiation (IR) along with green and red light to achieve the determined intensity level and emitting for measuring heart rate of the user. In one embodiment, the transmitter module 204 may use WDM splitters or combiners to separate different wavelength ranges and combine different wavelengths signals into one output channels respectively, as depicted in FIG. 7B.

Referring to FIG. 7B, a pictorial representation of the WDM is depicted. For example, scenario 721 of FIG. 7B depicts a scenario in which the WDM can be utilized as combiner or multiplexer to perform combination of diverse spectral bands for optimal results, the signals of different spectral bands are aggregated or combined into a single output channel. For instance, in circumstances where there are red and green light signals, a third output signal is created by aggregating or combining these two signals. As another example, scenario 722 of FIG. 7B depicts a scenario in which the WDM can be utilized as a splitter or de-multiplexer to separate different wavelength ranges, particularly the visible wavelengths under certain conditions. When the input signal has broad bandwidth, it may be divided into output components as required.

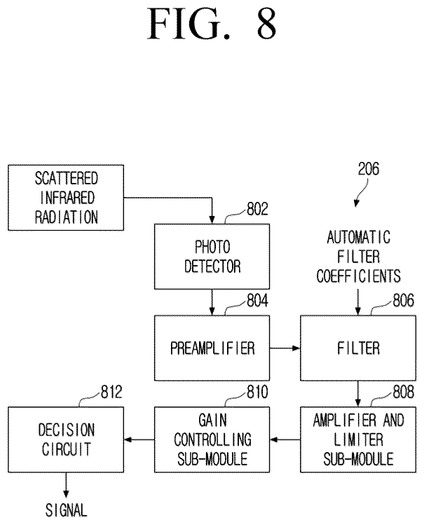

Referring again to FIG. 2, the system 200 may further include a receiver module 206 for receiving scattered IR radiation resulting from interaction of the transmitted IR radiation and visible light with user's skin condition based on the environment condition and providing the heart rate to user. In one embodiment, the receiver module 206 may include a plurality of sub-modules as depicted in FIG. 8.

Referring to FIG. 8, a block diagram of the receiver module 206 is depicted. The receiver module 206 may include a photo detector 802 for detecting and converting the scattered infrared radiation into electrical signals. The receiver module 206 may further include a preamplifier 804 for amplifying the electrical signal and a filter 806 for filtering the amplified signal based on filter coefficients. In one embodiment, the filter coefficients may depend on emitted wavelength. In another embodiment, the filter coefficients may depend on specific filtering requirements of the signal being processed and type of filter being used. After the signal has been filtered, additional processing may be performed to increase a strength of the signal and reduce any unwanted noise or distortion. For example, the receiver module 206 may use an amplifier and limiter sub-module 808 for improving quality of the filtered signal. In embodiments, the amplifier and limiter sub-module 808 may include an amplifier configured to increase the strength of the filtered signal to an appropriate level for further processing, and a limiter configured to ensure that the signal does not exceed certain amplitude to prevent any unwanted distortion or signal clipping. The receiver module 206 may further include a gain controlling sub-module 810 for improving signal-to-noise ratio, reducing distortion, and minimizing interference of the amplified signal, and a decision circuit 812 for processing the amplified signal and providing a signal suitable for the correlation module 208. In embodiments the decision circuit 812 may include at least one of low-pass filters and comparators.

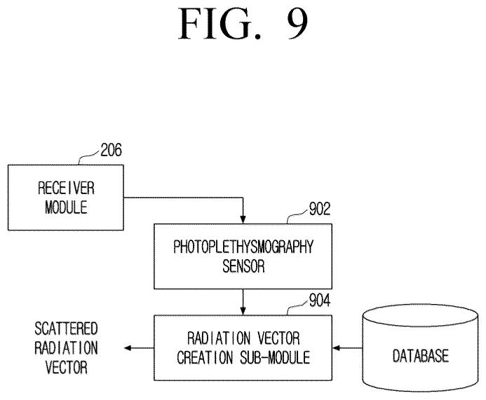

Referring again to FIG. 2, the system 200 may further include a correlation module 208 for determining scattered radiation vector from the received IR radiation. In one embodiment, the correlation module 208 may include a PPG sensor 902 and a radiation vector creation sub-module 904 as depicted in FIG. 9.

FIG. 9 is a block diagram of the correlation module 208, in accordance with one or more embodiments. As shown in FIG. 9, the PPG sensor 902 may be configured to receive scattered IR radiation from the receiver module 206 and further configured to determine amount of absorbed IR radiation and absorbed visible light such as green and red light. The radiation vector creation sub-module 904 may be configured to determine a scattered radiation vector.

The radiation vector creation sub-module 904 may be further configured to identify deviation by correlating amount of IR radiation absorption and visible light absorption with similar wavelength absorption corresponding to various skin attributes stored in the database 211 using a machine learning model. In one embodiment, the database 211 may be a multidimensional structure that stores attributes such as skin attributes, green light absorption, red light absorption, JR radiation absorption, age, and gender of different users. Table 3 shows an embodiment of information which may be stored in the database 211.

TABLE 3
Attributes which may be stored in the database

| Skin | | | | Heart Rate | Current Heart rate | |
|---|---|---|---|---|---|---|
| Attribute | Age | Gender | Weight | (Algorithmic) | (wearable) | Remark |
| [0.8, 0.3] | 22 | M | 95 kg | 96 | 95 | User is sweating |
| | | | | | | due to light walk |
| [0.6, 0.6] | 24 | F | 55 kg | 97 | 95 | User with dry skin is |
| | | | | | | jogging lightly and |
| | | | | | | sweating moderately |
| [0.4, 0.2] | 21 | M | 70 kg | 127 | 132 | User is engaged in |
| | | | | | | high-intensity exercise |
| | | | | | | and sweating heavily |
| [0.7, 0.9] | 45 | M | 80 kg | 82 | 71 | User with dry skin is |
| | | | | | | walking lightly and |
| | | | | | | experiencing little sweat |

As shown in Table 3, the database 211 may store information such as skin attribute [0.8, 0.3], algorithmic heart rate (96) computed using embodiments of the present disclosure, and current heart rate from the wrist watch (95) for a male user of age 22 and weight 95 kg when the user is sweating due to light walk. Similarly, the database 211 may store the skin attribute, algorithmic heart rate computed according to embodiments of the present disclosure, and current heart rate from the wrist watch for different users under different conditions.

In an embodiment, the radiation vector creation sub-module 904 may calculate the scattered radiation vector using Equation 2 below, and the algorithmic heart rate may be calculated using Equation 3 below:

$$Equation 2$$

$$Scattered Radiation Vector ( SRV ) = ( ( K [ 0 ] + K [ 1 ] - 1 ) / 10 ) + 1 )$$

$$Equation 3$$

$$Algorithmic heart rate = INT ( ( K [ 0 ] + K [ 1 ] - 1 ) / 10 ) + 1 ) * ( CHR )$$

In Equation 2 and Equation 3 above, K may denote the skin attribute K[0] may denote a dryness or a moisture content of the skin, and K[1] may denote an oil content of the skin, and CHR may denote a current heart rate calculated by the wrist watch without considering any factor.

Table 4 shows an embodiment of computing the scattered radiation vector and utilizing the scattered radiation vector for computing the algorithmic heart rate from the current heart rate stored in the database 211, and actual heart rate for different skin attributes.

TABLE 4
Determination of scattered radiation vector, algorithmic heart
rate, and actual heart rate for different skin attributes

| | | Algorithmic Heart | Actual Heart |
|---|---|---|---|
| Skin Attribute | Scattered Radiation Vector | Rate | Rate |
| [0.8, 0.3] | ((0.8 + 0.3 − 1)/10) + 1 = 1.01 | 95*1.01 = 96 | 96 |
| [0.6, 0.6] | ((0.6 + 0.6 − 1)/10) + 1 = 1.02 | 95*1.02 = 97 | 97 |
| [0.4, 0.2] | ((0.4 + 0.2 − 1)/10) + 1 = 0.96 | 132*0.96 = 127 | 125 |
| [0.7, 0.9] | ((0.7 + 0.9 − 1)/10) + 1 = 1.26 | 71*1.16 = 82 | 80 |

As can be seen in Table 4, there may be a very small difference between the algorithmic heart rate, and actual heart rate.

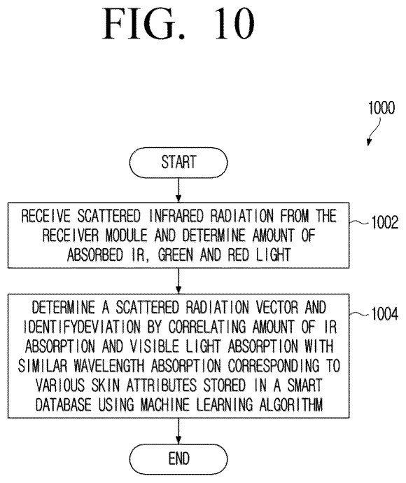

Referring to FIG. 10, a flow diagram showing a method of determining scattered radiation vector is depicted. As shown in FIG. 10, scattered IR radiation may be received from the receiver module 206, and an amount of absorbed IR radiation and absorbed visible light such as green and red light may be determined at operation 1002. Thereafter, a scattered radiation vector may be determined and deviation may be identified at operation 1004, by correlating amount of IR radiation absorption and visible light absorption with similar wavelength absorption corresponding to various skin attributes stored in the database, for example using a machine learning algorithm.

Referring again to FIG. 2, the system 200 may further include a detection module 210. The detection module 210 may be configured to determine wavelength of the visible light to be adjusted based on the scattered radiation vector and sending to the transmitter module 204 for improving the accuracy of the heart rate measurement based on the environmental condition. In one embodiment, the detection module 210 may determine red light and green light coefficients based on the scattered radiation vector and generating a suitable intensity level of the visible light to emit based on determined red light coefficient and green light coefficient using a coefficient generation neural network. Table 5 show the different red light and green light coefficients for different scattered radiation vectors considering the normalization factor 1000.

TABLE 5
Different red light and green light coefficients
for different scattered radiation vectors

| Scattered | | |
|---|---|---|
| Radiation Vector | Red Light Coefficient | Green Light Coefficient |
| 1.01 | (abs(3907621 − 3908003) * 1.01)/ | No Change |
| | 1000 = 0.39 | |
| 1.02 | (abs(3908037 − 3907667) * 1.02)/ | (abs(2142613 − 2141085) * |
| | 1000 = 0.38 | 1.02)/1000 = 1.56 |
| 0.96 | No Change | (abs(2095017 − 2093280) * |
| | | 0.96)/1000 = 1.67 |
| 1.16 | (abs(3910110 − 3910622) * 1.16)/ | No Change |
| | 1000 = 0.59 | |

In case the deviation is identified, the red light coefficient and green light coefficient may be calculate using the Equation 4 below:

Red:Green Light Coefficient=(Red:Green Light Absolute Difference*Scattered radiation vector)/Normalization Factor Equation4

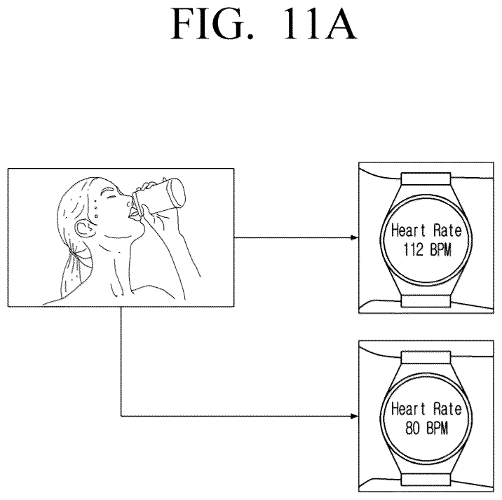

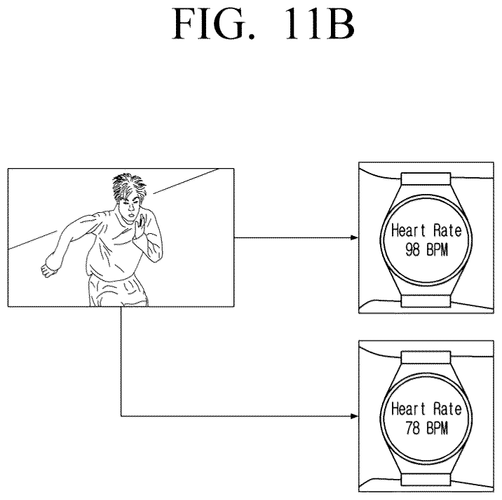

Referring to FIG. 11A, a first use case of improving heart rate measurement accuracy of the wearable device is depicted. As shown in FIG. 11A, the user may attempt to measure the heart rate in a humid environment. However, a heart rate measurement performed according to a comparative example may provide a heart rate measurement 1111 including a false heart rate reading caused by infrared radiation being absorbed by the sweat present due to humid weather, potentially causing the user to become distressed or worried. In contrast, embodiments of the present disclosure may provide a heart rate measurement 1112 including an accurate heart rate reading due to adjustment in wavelength/intensity of at least one of IR radiation and light emitted by the PPG sensor 308 according to the humid environment. For example, embodiments of the present disclosure may allow the wavelength of IR radiation to be adjusted to improve the accuracy of the heart rate measurement 1112 by determining an amount of absorbed IR radiation based on environmental conditions, determining the suitable wavelength to be transmitted based on the amount of scattered IR radiation and the attributes of the user's skin. Embodiments of the present disclosure may continuously monitor the environmental conditions to assess the effectiveness of the adjusted wavelength.

Referring to FIG. 11B, a second use case of improving heart rate measurement accuracy of the wearable device is depicted. As shown in FIG. 11B, the user may attempt to measure the heart rate while performing an intense work out. However, a heart rate measurement performed according to a comparative example may provide a heart rate measurement 1121 including a false heart rate reading due to IR radiation being absorbed by the sweat caused by the work out, potentially causing the user to become distressed or worried. In contrast, embodiments of the present disclosure may provide a heart rate measurement 1122 including an accurate heart rate reading due to adjustment in wavelength/intensity of at least one of IR radiation and light emitted by the PPG sensor 308 according to the environment and intense work out.

It has thus been seen that the system and method for improving heart rate measurement accuracy of the wearable device according to the present disclosure achieve the purposes discussed above. Although certain embodiments of the disclosure are described in detail above, the scope of the disclosure is not limited the described embodiments. Various changes and modifications shall be construed to belong to the scope of the disclosure as defined by the attached claims.

What is claimed is:

1. A method for adjusting heart rate measurement accuracy of a wearable device, the method comprising:
obtaining, by a data module, a plurality of pieces of data including user activity data, user information, and environment conditions related to a user from a plurality of data sources;
determining a skin attribute of the user based on the obtained plurality of pieces of data;
emitting, by a transmitter module, infrared (IR) radiation and visible light at an intensity level for measuring a heart rate of the user;
receiving, by a receiver module, scattered IR radiation resulting from interaction of the transmitted IR radiation and the transmitted visible light based on a skin condition of the user and the environment conditions;
determining, by a correlation module, a scattered radiation vector based on the scattered IR radiation;
determining, by a detection module, a wavelength of the visible light to be adjusted based on the scattered radiation vector; and
adjusting, by the transmitter module, the wavelength of the visible light.

2. The method as claimed in claim 1, wherein the user activity data comprises physical activity data of the user,
wherein the user information comprises at least one of electronic health record data, disease information, age, gender, and location of the user, and
wherein the environment conditions comprise a temperature and a humidity of an environment of the user.

3. The method as claimed in claim 1, wherein the plurality of data sources comprise at least one of an electronic health record data of the user, a photoplethysmography sensor, a temperature sensor, an accelerometer, and a gyroscope.

4. The method as claimed in claim 1, further comprising:
determining, using an input module included in the data module, user demographics based on the user information obtained from the plurality of data sources; and
determining, using a skin attribute extraction module included in the data module, the skin attribute based on at least one of the user demographics obtained from the input module, and electronic health record data, physical activity data, temperature data, and humidity data obtained from the plurality of data sources.

5. The method as claimed in claim 4, wherein the determining the skin attribute of the user comprises:
determining, using a skin type vector determining sub-module, a skin type vector of the user based on at least one of the electronic health record data and historical data provided by the user, wherein the skin type vector indicates at least one of a dry skin magnitude, an oily skin magnitude, and a normal skin magnitude based on the skin condition of the user;
determining, by a sweat amount determining sub-module, a sweat amount based on the user demographics, the physical activity data, the temperature data, and the humidity data using a machine learning model; and
determining, by a vector merger, the skin attribute by merging the skin type vector determined by the skin type vector determining sub-module and the sweat amount determined by the sweat amount determining sub-module.

6. The method as claimed in claim 4, wherein the user demographics, the plurality of pieces of data, and the skin attribute of the user are stored in a database.

7. The method as claimed in claim 1, further comprising:
receiving, using a controller included in the transmitter module, input from the data module and the detection module and determining the intensity level of the visible light based on a red light coefficient and a green light coefficient, wherein the visible light includes red light and green light; and
emitting, using a modulator included in the transmitter module, IR radiation along with the green light and the red light based on the determined intensity level.

8. The method as claimed in claim 1, further comprising at least one of:
separating different wavelengths of light using a wavelength division multiplexing (WDM) splitter; and
combining the different wavelengths of light into one output channel using a WDM combiner.

9. The method as claimed in claim 1, further comprising:
detecting the scattered IR radiation and converting the scattered IR radiation into an electrical signal using a photo detector;
amplifying the electrical signal using a preamplifier;
filtering the electrical signal based on filter coefficients using a filter, wherein the filter coefficients depend on the wavelength of the visible light;
improving a quality of the electrical signal using at least one of an amplifier and a limiter;
improving a signal-to-noise ratio, reducing a distortion, and minimizing an interference of the electrical signal using a gain controlling sub-module;
processing the electrical signal using a decision circuit comprising at least one of a low-pass filter and a comparator; and
providing the electrical signal to the correlation module.

10. The method as claimed in claim 1, wherein the determining the scattered radiation vector comprises:
receiving, by a photoplethysmography sensor, the scattered IR radiation from the receiver module and determining amount of absorbed IR radiation and an amount of absorbed visible light, wherein the absorbed visible light comprises at least one of green light and red light; and
determining, by a radiation vector creation sub-module, the scattered radiation vector and identifying a deviation by correlating an amount of IR radiation absorption and visible light absorption with similar wavelength absorption corresponding to a plurality of skin attributes stored in a database using a machine learning model.

11. The method as claimed in claim 1, further comprising determining, by the detection module, a red light coefficient and a green light coefficient based on the scattered radiation vector; and
determining the intensity level of the visible light based on the determined red light coefficient and the determined green light coefficient using a coefficient generation neural network.

12. A system for adjusting heart rate measurement accuracy of a wearable device, the system comprising:
a data module configured to obtain a plurality of pieces of data comprising user activity data, user information, and environment conditions from a plurality of data sources and determine a skin attribute of a user based on the obtained plurality of pieces of data;
a transmitter module configured to emit infrared (IR) radiation along with visible light at an intensity level for measuring a heart rate of the user;
a receiver module configured to receive scattered IR radiation resulting from interaction of the transmitted IR radiation and the transmitted visible light with based on a skin condition of the user and the environment conditions;
a correlation module configured to determine a scattered radiation vector based on the scattered IR radiation; and
a detection module configured to determine wavelength of the visible light to be adjusted based on the scattered radiation vector,
wherein the transmitter module is further configured to adjust the wavelength of the visible light.

13. The system as claimed in claim 12, wherein the user activity data comprises user physical activity data,
wherein the user information comprises at least one of electronic health record data, disease information, age, gender, and location of the user, and
wherein the environment conditions comprise at least one of a temperature and a humidity of an environment of the user.

14. The system as claimed in claim 12, wherein the plurality of data sources comprise at least one of an electronic health record data of the user, a photoplethysmography sensor, a temperature sensor, an accelerometer, and a gyroscope.

15. A non-transitory computer readable recording medium storing instructions which, when executed by at least one processor of a device for heart rate measurement of a wearable device, cause the device to:
obtain, by a data module, a plurality of pieces of data comprising user activity data, user information, and environment conditions related to a user from a plurality of data sources;
determine a skin attribute of the user based on the obtained plurality of pieces of data;
emit, by a transmitter module, infrared (IR) radiation along with visible light at an intensity level for measuring heart rate of the user;
receive, by a receiver module, scattered IR radiation resulting from interaction of the emitted IR radiation and the emitted visible light based on a skin condition of the user and the environment condition;
determine, by a correlation module, a scattered radiation vector based on the scattered IR;
determine, by a detection module, a wavelength of the visible light to be adjusted based on the scattered radiation vector; and
adjust, by the transmitter module, the wavelength of the visible light.

16. The non-transitory computer readable recording medium as claimed in claim 15, wherein the user activity data comprises physical activity data of the user,
wherein the user information comprises at least one of electronic health record data, disease information, age, gender, and location of the user, and
wherein the environment conditions comprise a temperature and a humidity of an environment of the user.

17. The non-transitory computer readable recording medium as claimed in claim 15, wherein the plurality of data sources comprise at least one of an electronic health record data of the user, a photoplethysmography sensor, a temperature sensor, an accelerometer, and a gyroscope.

18. The non-transitory computer readable recording medium as claimed in claim 15, storing instructions which cause the device to:
determine, using an input module included in the data module, user demographics based on the user information obtained from the plurality of data sources; and
determine, using a skin attribute extraction module included in the data module, the skin attribute based on at least one of the user demographics obtained from the input module, and electronic health record data, physical activity data, temperature data, and humidity data obtained from the plurality of data sources.

19. The non-transitory computer readable recording medium as claimed in claim 15, storing instructions which cause the device to:
determine, using a skin type vector determining sub-module, a skin type vector of the user based on at least one of the electronic health record data and historical data provided by the user, wherein the skin type vector indicates at least one of a dry skin magnitude, an oily skin magnitude, and a normal skin magnitude based on the skin condition of the user;
determine, by a sweat amount determining sub-module, a sweat amount based on the user demographics, the physical activity data, the temperature data, and the humidity data using a machine learning model; and
determine, by a vector merger, the skin attribute by merging the skin type vector determined by the skin type vector determining sub-module and the sweat amount determined by the sweat amount determining sub-module.

20. The non-transitory computer readable recording medium as claimed in claim 15, wherein the user demographics, the plurality of pieces of data, and the skin attribute of the user are stored in a database.
